(19) United States
(12) Patent Application Publication
(10) Pub. No.: US 2003/0191493 A1
(43) Pub. Date: Oct. 9, 2003

(54) Device for clot retrieval and distal protection

(76) Inventors: Gordon H. Epstein, Fremont, CA (US); Randall T. Higashida, Tiburon, CA (US)

Correspondence Address: FLEHR HOHBACH TEST, ALBRITTON & HERBERT LLP, Suite 3400, Four Embarcadero Center, San Francisco, CA, 94111-4187, US

(21) Appl. No.: 10/118,390

(22) Filed: Apr. 5, 2002

(51) Int. Cl. A61M 29/00

(52) U.S. Cl. 606/200

(57) ABSTRACT

A device for retrieving clot material and distal protection within a blood vessel having a wall defining a lumen in the body. The device comprises an elongated tubular member having proximal and distal extremities and having a longitudinal axis. An expansile member having proximal and distal extremities is carried by the distal extremity of the elongated tubular member and is movable between radially contracted and expanded configurations. A deformable membrane at least partially covers the expansile member in the radially expanded configuration. A guide member is coupled to the distal extremity of the expansile member and has proximal and distal ends. A handle assembly is carried by the proximal extremity of the elongated tubular member for moving said expansile member between radially contracted and expanded configurations. The handle assembly is detachable from the proximal extremity of the elongated tubular member.

### FIELD OF THE INVENTION

This invention relates to a device for retrieving clot and other foreign material from blood vessels and protecting the distal vasculature during invasive procedures.

### BACKGROUND OF THE INVENTION

Distal protection of the vasculature during invasive procedures as well as retrieval of clot debris and other matter therefrom has heretofore been accomplished. These interventional therapies can be performed either individually or concomitantly and are appropriate for the coronary, peripheral and cerebral vasculature. Various devices and methods have been utilized heretofore, however, they have all been plagued with deficiencies. Specifically, the relatively small size of the distal coronary vessels as well as the smaller size and tortuosity of the cerebral vasculature has made traversing and navigating the same during therapeutic intervention often traumatic and/or impossible. There is, therefore, a need for a device and method for protecting the distal vasculature during invasive procedures in the human body as well as percutaneously retrieving embolic or other clot material therefrom which overcomes the deficiencies of prior art devices and methods.

### SUMMARY OF THE INVENTION

In general, it is an object of the present invention to provide a device for retrieving clot material and protecting distal vasculature during interventional procedures which is sized and structured in order to facilely navigate small and tortuous blood vessels in the human body.

Another object of the invention is to provide a device of the above character which can be easily and reliably deployed and used.

Another object of the invention is to provide a device of the above character which offers superior trackability during deployment.

Another object of the invention is to provide a device of the above character which is cost effective.

Additional objects and features of the invention will appear from the following description from which the preferred embodiments are set forth in detail in conjunction with the accompanying drawings.

### BRIEF DESCRIPTION OF THE DRAWINGS

FIG. 1 is a cross sectional view of a clot retrieval and distal protection device of the present invention having the expansile member in the radially contracted configuration.

FIG. 2 is a cross sectional view of the device of FIG. 1 with the expansile member in the radially expanded configuration.

FIG. 3 is a side elevational view of the distal end of the device of FIG. 2

### DETAILED DESCRIPTION OF THE INVENTION

In general, the device of the present invention is used for retrieval of clot material and other matter and distal protection within a blood vessel having a wall defining a lumen in the body. The clot retrieval and distal protection device comprises an elongated tubular member having proximal and distal extremities and having a longitudinal axis. An expansile member having proximal and distal extremities is carried by the distal extremity of the elongated tubular member and is movable between radially contracted and expanded configurations. A deformable membrane at least partially covers the expansile member in the radially expanded configuration. A guide member is coupled to the distal extremity of the expansile member and has proximal and distal ends. A handle assembly is carried by the proximal extremity of the elongated tubular member for moving said expansile member between radially contracted and expanded configurations. The handle assembly is detachable from the proximal extremity of the elongated tubular member.

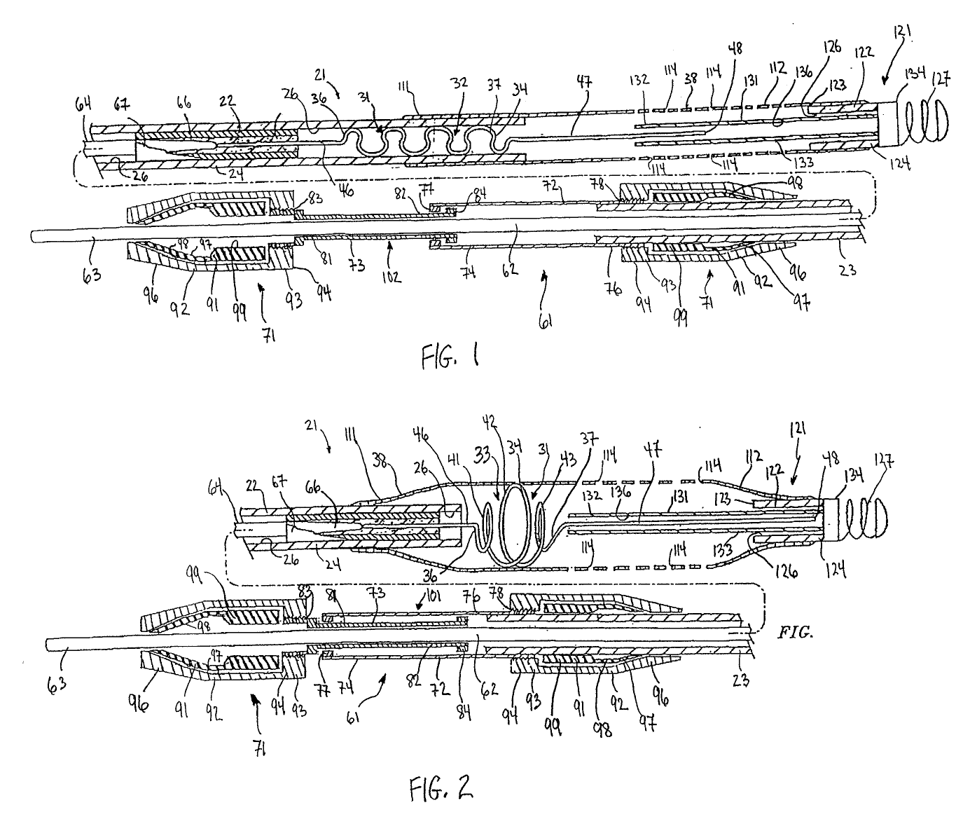

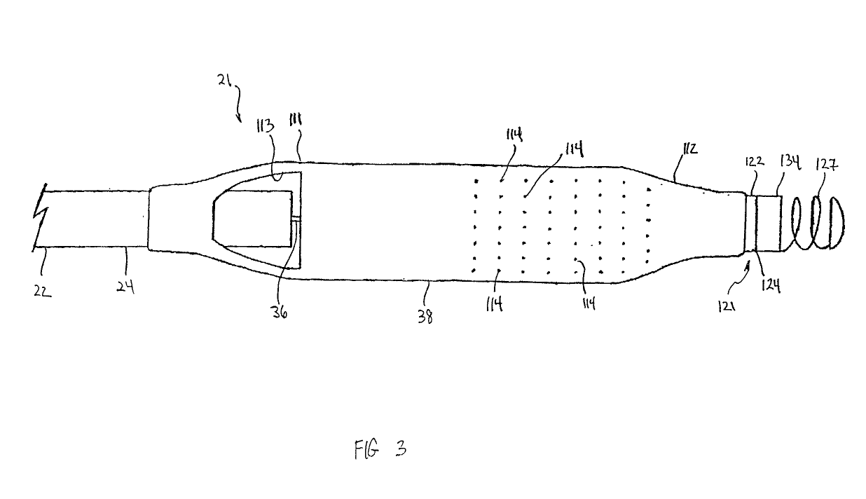

More specifically, as shown in FIGS. 1-2, the expansile device 21 of the present invention comprises a first elongate tubular member 22, preferably a flexible elongate tubular member 22, formed of a suitable plastic material, preferably a cast thermoset material such as polyimide. The inner and/or outer surfaces of the polyimide material may be coated with a lubricious material such as Teflon™. Alternatively, the thermoset material may be a polyimide-Teflon™ composite in order to provide the desired lubricious inner and outer surfaces. The first flexible elongate tubular member 22 has proximal and distal extremities 23 and 24 with a longitudinal axis extending from the proximal 23 to the distal extremity 24 and is provided with a first lumen 26 which is circular in cross-section and which, as shown, may be centrally disposed, extending from the proximal extremity 23 to the distal extremity 24.

Flexible elongate tubular member 22 is of a suitable size, as for example having an outer diameter ranging approximately 0.005-0.040 inches, preferably approximately 0.001″, and a suitable length, as for example, ranging from 2 to 60 centimeters, preferably being approximately 40 centimeters ±1 centimeter. First lumen 26 may have an inside diameter of approximately 0.004-012″, preferably approximately 0.008″.

It should be appreciated that the elongate tubular member may be formed of other materials such as stainless steel hypotube, Nitinol or Pebax™ and still fall within the purview of the present invention. For example, the tubular member may be constructed of hypotube or another suitable material which provides optimal torquability and trackability. Alternatively, the elongate tubular member may be a combination of the foregoing, being provided with a proximal extremity constructed of hypotube and appropriately coupled to a distal extremity constructed of a plastic material.

A tip guide (not shown) is slidably carried by polyimide tubular member 22 for use as hereinafter discussed. The tip guide is constructed of 1/16″ RNF 100 Shrink tubing, has a longitudinal axis and a length of approximately 20 millimeters. In addition, the distal portion of the tip guide is provided with a larger, non-shrunk end.

Expansile means in the form of an expansile assembly 31 is carried by distal extremity 24 of flexible elongate tubular member 22 and is movable between radially contracted and expanded configurations 32 and 33. Expansile assembly 31 includes an expansile member 34 having proximal and distal extremities 36 and 37 and a deformable membrane 38 which at least partially covers expansile member 32. As shown in FIG. 2, expansile member 34 is in a form having a complex geometrical configuration, preferably a ellipsoidal, helical or bi-conical coil configuration 39, when in the free, unconstrained state. As hereinafter discussed, helical coil 39 is formed of a suitable material such as a shape memory or superelastic material which can be elongated, contracted or constrained without permanent deformation but, at body or room temperature, when freed or unconstrained returns to the memorized helical coil configuration 39 to which it has been annealed. One material found to be particularly suitable for such an application is a nickel/titanium alloy wire, often called Nitinol wire.

The correctly annealed and configured helical coil 39 comprises a plurality of substantially coaxial circular turns, loops or coils creating, preferably, a proximal coil, loop or turn 41, a middle coil, turn or loop 42 and a distal coil, turn or loop 43 as shown in FIG. 2. Proximal, middle and distal coils 41, 42 and 43 are generally non-planar with respect to one another. At least a portion of proximal coil 41 and a portion of distal coil 43 each lie in a plane that is generally parallel to one another and generally perpendicular to the longitudinal axis of flexible elongate tubular member 22. Middle coil 42 is non-planar and helical as it connects proximal and distal coils 41 and 43 so that the unconstrained or free helical coil 39 assumes a substantially ellipsoidal or bi-conical shape.

Middle coil 42, when freed or unconstrained, has a suitable diameter ranging from 1-12 millimeters, preferably, greater than or equal to 12 millimeters. As hereinafter discussed, during deployment, middle coil 42 is partially flattened and constrained by interacting with membrane 38 in order to span the lumen of a vessel. Proximal and distal coils 41 and 43 are of approximately equal size and diameter ranging from 0.9-12 millimeters, preferably 4-6 millimeters. Unconstrained helical coil 39 configuration has a distance from proximal 41 to the distal 43 coil of approximately 2-8 millimeters. As hereinafter discussed, helical coil 39 is retracted into flexible elongate tubular member 22 to obtain the de-deployed configuration wherein the contracted, constrained diameter corresponds to the approximate diameter of the Nitinol wire used to construct expansile member 34, ranging from 0.001-0.010″.

Expansile member 34 is also provided with a straight segment or portion 46 of Nitinol wire proximal to helical coil 39 having a length of approximately 20 millimeters ±18 millimeters and a straight distal leg 47 of Nitinol wire extending distal of the distal coil 43 for a length of approximately 6 millimeters ±5 millimeters. Distal leg 47 carries a distal tip 48 which determines the range of distal motion of expansile member 34 during movement of the same from radially contracted 32 to expanded configuration 33 as hereinafter described.

Handle assembly or deployment means 61 includes a push-pull element or member 62, preferably in the form of a wire 62 with proximal and distal extremities 63 and 64, which is slidably disposed in and extends through first lumen 26 of flexible elongate tubular member 22 as hereinafter discussed. Push-pull member 62 is formed of a suitable material such as stainless steel or Nitinol in order optimize column strength and torque transmission and is provided with a suitable diameter ranging from approximately 0.001″-0.015″, preferably approximately 0.007″. In order to provide for optimal torque transmission after being bonded to the Nitinol expansile member 34 as hereinafter discussed, distal extremity 64 of push-pull wire 62 is provided with a ground tapered portion or segment 66. Tapered segment 66 has a length ranging from approximately 0.5 centimeters to 2 centimeters.

A hypotube connector 67 is provided for joining tapered portion 66 of push-pull wire 62 to proximal straight portion 46 of the Nitinol wire. Hypotube connector 67 has a length ranging from approximately 0.5 centimeters to 5 centimeters (preferably, approximately 1 centimeter), an inner diameter ranging from approximately 0.001″-0.010″ (preferably, approximately 0.005″) and an outer diameter ranging from approximately 0.002″-0.012″. During manufacture, tapered portion 66 of push-pull wire 62 is inserted into one end of hypotube connector 67 and the proximal end of straight portion 46 of the Nitinol wire is inserted into the opposite, distal end of connector 67 whereupon all are bonded together within hypotube connector 67 utilizing a suitable adhesive such as Loctite™648. It should be appreciated that connector 67 can also be constructed of any other appropriate material such as polyimide, Nitinol or other flexible plastic material.

Handle assembly 61 includes means 71 for enabling the same to be reversibly coupled and uncoupled or attached and detached from both proximal extremity 23 of elongated tubular member 22 and proximal extremity 63 of wire 62. In this regard, first and second, or outer and inner, slidably and coaxially nested stop tubes 72 and 73 are provided which are formed of an appropriate material having an appropriate thickness, such as stainless steel hypotube. First stop tube 72 has proximal and distal ends 74 and 76, a length ranging from approximately 1 centimeter to 10 centimeters, preferably approximately 4 centimeters, and an inner diameter which is slightly greater than the outer diameter of flexible elongate tubular member 22. Proximal end 74 of first stop tube 72 carries a bushing 77 secured thereto by any suitable means such as an adhesive as seen in FIGS. 1-2. Distal end 76 of first stop tube 72 is provided with an externally threaded segment or region 78 of appropriate length as hereinafter described.

Second stop tube 73 ranges from approximately 1 to 10 centimeters, preferably being approximately 4 centimeters, in length. The proximal end 81 of second tube 73 is also provided with an externally threaded segment 83 of appropriate length. In addition, the distal end 82 of second tube 73 is provided with a external annular ring or collar 84 which is secured thereto by any suitable means as hereinbefore described. The external diameter of collar 84 is slightly less than the internal diameter of first stop tube 72 while the outer diameter of second stop tube 73 is, in turn, slightly less than the internal diameter of collar 84.

First and second stop tubes 72 and 73 each carry a conventional collet 91 and collar 92 unit constructed of an appropriate material such as metal or plastic and threadedly coupleable thereto as seen in FIGS. 1-2. In this regard, first end 93 of collar 92 carries an inwardly extending, internally threaded flange 94 by which collar 92 can be coupled to threaded region 78 of distal end 76 of first stop tube 72 or threaded region 83 of proximal end 81 of second tube 73. At least the inner surface of second end 96 of collar 92 is inwardly tapered away from first end 93 thereby presenting a conical configuration. Collet 91 comprises a conventional conical sleeve 97 which is provided with a plurality of longitudinal slits producing a plurality of circumferentially disposed tines 98. Sleeve 97 is concentrically, rotatably disposed within collar 92 as is traditional in the art. In this regard, collet 91 is provided with an inwardly extending flange 99 at one end which is sized in order to seat against both flange 94 of first end 93 of collar 92 as well as respective distal and proximal ends 76 and 81 of first and second stop tubes 72 and 73. The conical, opposite end of sleeve 97 is compressibly contained within and restrained by the second end of collar 96 as seen in FIGS. 1-2 and as hereinafter described.

During coupling of handle assembly 61 to proximal extremity 23 of flexible elongate tubular member 22 and to proximal extremity 63 of wire 62 as hereinbefore described, proximal extremity 23, with wire 62 extending proximal thereof, passes proximally through the distal collet 91 and collar 92 to become slidably disposed within distal end 76 of first stop tube 72 an appropriate distance. Proximal extremity 63 of wire 62 extends further proximally, through first and second stop tubes 72 and 73 and through the proximal collet 91 and collar 92 as seen in FIG. 1. Handle assembly 61 is secured in place upon flexible elongate tubular member 22 by rotating the distal collar 92 on collet 91 whereupon collar 92 is moved in a proximal direction thereby compressing tines 98 of conical sleeve 97 against flexible elongate tubular member 22 in order to grasp the same. In a similar fashion, second stop tube 73 is secured in place upon proximal extremity 63 of wire 62 using the proximal collet 91 and collar 92 unit.

Accordingly, proximal collet 91 and collar 92, with second stop tube 73 and wire 62 fixed relative thereto, is movable longitudinally of first stop tube 72 which has distal end 76 thereof secured to proximal extremity 23 of the elongate tubular member 22. Specifically, second stop tube 73 is movable from a forward-most position 101 where collar 84 carried thereby engages proximal extremity 23 and a rearward-most position 102 where collar 84 engages bushing 77 carried by proximal end 74 of first stop tube 72. The lengths of the first and second tubes 72 and 73 are selected so that the travel between the forward-most and rearward-most positions 101 and 102 ranges between 0.5 cm and 10 cm. In addition, it is apparent that the travel distance can be manipulated readily by loosening collets 91 and re-fixing the foregoing elements relative thereto.

As hereinbefore discussed, expansile assembly 31 also carries a deformable membrane 38 which is carried by and secured to distal extremity 24 of elongate tubular member 22 as shown in FIGS. 1-2. Membrane 38 is formed by being molded of polyethylene terephthalate (PET) extrusion and has a thickness ranging from approximately 0.00005-0.010 inches and, preferably of approximately 0.0001″, and which has a length of approximately 18 millimeters ±17 millimeters. The proximal end or portion 111 of membrane 38 is secured to distal extremity 24 of flexible elongate tubular member 22 using an appropriate material such as Loctite 496 adhesive so that the distal membrane tip 112 extends distal to the tip of distal extremity 24 of flexible elongate tubular member 22 and so that the distally extending portion of membrane tip 112 has a length, measured from the tip of distal extremity 24 of flexible elongate tubular member 22 to the distal end of membrane tip 112, of approximately 15 millimeters. Proximal portion 111 of membrane 38 is provided with a cutout which forms an orifice or window 113 therein as shown in FIG. 3. Orifice 113 is longitudinally tapered to have a proximal width ranging from approximately 0.5 to 1.5 millimeters, preferably approximately 1 millimeters, and a distal width ranging from approximately 1 to 4 millimeters, preferably approximately 3.5 millimeters. Membrane 38 also carries a plurality of micropores 114 distributed over the distal half thereof as shown in FIG. 3. Micropores 114 each range from approximately 1-300 microns in diameter, preferably being approximately 80 microns in diameter.

Expansile assembly 31 is provided with a guide assembly 121 comprising a leading or lead member 122 secured by being glued or similarly adhesively attached to distal end 112 of membrane 38 by laser welding, ultra-violet adhesives or epoxy as shown in FIGS. 1-2. Lead member 122 is formed of a suitable plastic material as, by way of example, a cast thermoset material such as polyimide or Pebax™. Lead member 122 has proximal and distal extremities 123 and 124, extends along a longitudinal axis from the proximal 123 to the distal extremity 124 and is provided with a lumen 126 which is circular in cross-section, centrally disposed and extends from the proximal extremity 123 to the distal extremity 124 thereof.

Lead member 122 is of a suitable size, as for example having an outer diameter ranging from approximately 0.005″-0.040″, preferably approximately 0.011″, and a suitable length, as for example ranging from approximately 1-20 millimeters, preferably being approximately 5 millimeters. Lumen 126 may have a diameter ranging from approximately 0.004″-0.012″, preferably approximately 0.008″. Distal end 112 of membrane 38 is secured to proximal extremity 123 of lead member 122 in an appropriate manner as hereinbefore described and as shown in FIGS. 1-2. Depending on the use to which device 21 is put and the trackability issues associated therewith, a conventional platinum floppy, coiled, pre-shaped or shapable wire tip 127 is appropriately secured to distal extremity 124 of lead member 122 and extends distal thereof.

Expansile assembly 31 is also provided with a guide member 131 which is coupled to proximal extremity 123 of leading member 122 by being glued or otherwise adhesively attached thereto. Guide member 131 is provided with proximal and distal ends 132 and 133 and is preferably constructed of polyimide or stainless steel hypotube having a length ranging from approximately 1-30 millimeters, preferably approximately 8 millimeters, an inner diameter ranging from approximately 0.005-010″ and an outer diameter of approximately 0.007″. Distal end 133 of guide member 131 is provided with a seal or cap 134 which is formed in any appropriate manner. In this regard, distal end 133 may be soldered closed or provided with a plug formed of a suitable material.

Distal end 133 of guide member 131 is adhesively, concentrically secured in an appropriate manner within lumen 126 of proximal extremity 123 of leading member 122 so that guide member 131 extends coaxially, proximal of leading member 122 and is thereby bound by, contained within and covered by distal end 112 of membrane 38.

With expansile member 34 in contracted configuration 32, distal tip 48 of distal leg 47 is slidably, coaxially disposed within the proximal-most region of the lumen 136 of guide member 131 as shown in FIG. 1. During assumption of the radially expanded configuration 33 by expansile member 34, distal leg 47 slides distally within lumen 136 of guide member 131 until distal tip 48 abuts seal 134 of distal end 133 of guide member 131 as shown in FIG. 2.

Operation and use of the expansile device 21 of the present invention may now be described in conjunction with the accompanying figures as follows.

Let it be assumed that a patient has sustained myocardial ischemia as a result of an coronary embolic or acute thrombotic obstruction. Let it further be assumed that the patient has undergone coronary angiography whereby the embolic or thrombotic obstruction has been localized within the distal, small and tortuous coronary vasculature and that the clinical decision has been made to extract the embolic clot percutaneously. Immediately after or in conjunction with the aforementioned procedure and with the patient appropriately sedated and sterilely prepped, a conventional coronary interventional guide catheter is introduced over a guidewire via an indwelling conventional sheath introducer into the femoral artery (or any other appropriate arterial access site). Under fluoroscopy, the guide catheter is passed in a conventional manner into the coronary arterial system and into the proximity of the embolic lesion so that the distal tip of the guide catheter is disposed just proximal to the lesion.

Prior to deployment, except for straight distal leg 47, expansile member 34 is fully or completely retracted within distal extremity 24 of flexible elongate tubular member 22 causing it to assume contracted configuration 32. Membrane 38 is conventionally folded or pleated in a manner familiar in the art. Insertion of device 21, with handle assembly 61 secured thereto as hereinbefore described, into a conventional guide catheter (not shown) is facilitated by using the tip guide (not shown) carried by polyimide tubular member 22 as hereinbefore discussed. Prior to inserting device 21 into the guide catheter, the operator slides the tip guide distally, from the middle of polyimide tubular member 22 to distal extremity 24 thereof. When the distal end of the tip guide is disposed slightly distal to distal extremity 24 of polyimide tubular member 22 and membrane 38 carried thereby, the distal end of the tip guide is frictionally fit into the guide catheter. Distal extremity 24 of elongate tubular member 22 can then be easily and atraumatically introduced through the proximal opening of the guide catheter and advanced distally therein until device 21 is aptly disposed in the guide catheter in the blood vessel as hereinbefore discussed. By not relying on the tip of distal extremity 24 of device 21 to function as the leading edge while passing into the guide catheter, the integrity of membrane 38 carried thereby is maintained.

Once appropriately disposed in the guide catheter, device 21 is passed distally, in a manner similar to that used with a guidewire, until it exits the distal end of the guide catheter in the target vessel and traverses the embolic lesion. Appropriately disposed distal of the lesion, device 21 is poised for deployment.

Deployment of device 21 is accomplished by using handle assembly 61 to advance proximal collet 91, collar 92 and second stop tube 73 distally, away from rearward position 102 and into forward position 101. Concomitantly, push-pull wire 62 is advanced distally, urging expansile member 34 distally out of lumen 26 of flexible elongate tubular member 22, into membrane 38. As soon as distal extremity 37 of expansile member 34 clears lumen 26, it begins an attempt to expand into its shape memory, predetermined, or free configuration which corresponds to ellipsoidal, helical coil configuration 39. However, as hereinafter discussed, expansile member 34 is prevented from fully expanding into its free shape configuration as a result of membrane 38 partially constraining the expansion process.

More specifically, distal coil 43 operates to expand membrane 38 initially to a small degree. This initial process avoids sudden gross distortion of membrane 38. As soon as expansile member 34 moves further distally out of lumen 26 and expands into membrane 38, expansion proceeds with middle coil 42 causing membrane 38 to expand to its desired size. Proximal coil 41 expands last, to centralize and stabilize the configuration so that straight segment 46 is centered substantially with respect to middle coil 42 and the now fully expanded membrane 38.

Throughout the deployment process, as expansile member 34 is expanding and seeking its memorized configuration 39 it has a tendency to rotate in a leftward or counter-clockwise direction. This torque requires that the expansile member 34 be stabilized in order for it to operatively expand within, and without damaging, membrane 38 as. Stabilization during deployment is provided and controlled by distal leg 47 sliding distally within lumen 136 of guide member 131 until distal tip 48 abuts seal 134 of distal end 133 of guide member 131. This process prevents expansile member 34 from rotating into a lateral position relative to straight portion 46.

In addition, during expansion, as distal tip 48 abuts seal 134 membrane 38 simultaneously exerts a proximal longitudinal force upon guide member 131 due to the relationship of guide member 131 to lead member 122 and the attachment of membrane 38 to the latter. This causes guide member 131 to transmit the proximal force to distal leg 47 and thereby to constrain coil 34, thus exerting counteractive or countervailing forces on the expanding expansile member 34 which is seeking its memorized, unconstrained configuration 39. Thus, membrane 38 does not expand passively. Rather, expanding coil 34 forcibly expands membrane 38 which, in conjunction with the opposing forces by membrane 38 upon coil 34, cause the non-planar turns or coils 41, 42 and 43 of expansile member 34 to assume a substantially planar or disk-like configuration 33 within membrane 38 and which, when expanded, is generally perpendicular to the longitudinal axis of first flexible elongate tubular member 22. Expansile member 34, when so deployed into this constrained, partially expanded configuration, is sufficiently rigid and robust so as to provide a supporting framework for membrane 38 to keep a portion of it taut and capable of spanning and substantially occluding the lumen of the vessel in which device 21 is deployed. In fully deployed configuration 33, any perfusion remaining distal to the lesion (and proximal to expansile member 34) is maintained by blood preferentially entering orifice 113 in membrane 38 and exiting via distal micropores 114.

In order to capture the embolism, device 21 in fully deployed configuration 33 is pulled proximally whereby clot also enters orifice 113 in membrane 38. Too large to exit micropores 114, the clot is captured within the interior of membrane 38 while blood continues to exit therefrom. Subsequent to capture, device 21 is pulled further proximally until is abuts against the distal tip of the guide catheter. Proximal extremity 36 of expansile member 34 with overlying orifice 113 of membrane 38 must then be retracted partially into the guide catheter so that device 21 containing clot material can be extracted. Accordingly, the physician de-deploys expansile assembly 31 by moving handle assembly 61 in a reverse manner to retract proximal collet 91, collar 92 and second stop tube 73 proximally, away from forward position 101 and into rearward position 102. Concomitantly, push-pull wire 62 is pulled proximally, urging expansile member 34 proximally into lumen 26 of flexible elongate tubular member 22 and into contracted configuration 32. Membrane 38 is prevented from assuming it fully un-expanded or folded configuration because of clot contained therein. Nonetheless, by retracting proximal extremity 36 of expansile member 34 partially into guide catheter as hereinbefore described, orifice 113 is occluded and clot contained within membrane 38 can be effectively contained and extracted by pulling the guide catheter and device 21 simultaneously (as a unit) out of the vessel, out of the sheath introducer and, thus, out of the patient.

In another method of use of the present invention, device 21 is used to afford distal protection in conjunction with interventional maneuvers such as angioplasty, percutaneous atherectomy, stent placements and similar procedures with which fracture and distal showering of thrombotic or atheromatous material is associated. The operation and use of device 21 for distal protection is similar to that hereinbefore described in conjunction with clot extraction. The primary difference is that, once device 21 is fully deployed distal to a lesion which is to be treated with, as by way of example, angioplasty, a conventional angioplasty catheter is advanced into the lesion over flexible elongate tubular member 22 which thereby functions as the guidewire for the angioplasty catheter. In this regard, and in order for the angioplasty catheter to pass over flexible elongate tubular member 22, handle assembly 61 must be uncoupled and removed from proximal extremity 23. This is effected by reversing the process of coupling hereinbefore described. Thus, both collars 92 are loosened thereby releasing the respective grasps of proximal and distal collets 91 on push-pull member 62 and proximal extremity 23. In this manner, the entire handle assembly 61 is slid proximally, off of flexible elongate tubular member 22 and push-pull member 62 leaving the same receptive to a coaxially and slidably disposed overlying angioplasty catheter. After passing the proximal extremity of the angioplasty catheter distal to the proximal extremity of device 21, handle assembly 61 may be re-coupled as necessary for operation thereof. Thereafter, the remainder of the deployment and de-deployment of device 21 is as hereinbefore described. Similarly, removal of the angioplasty catheter over device 21 is effected by reversing the coupling process of handle assembly 61.

It is apparent from the foregoing that there has been provided a device for retrieving clot material or other foreign matter, thus restoring perfusion, and distal protection within a blood vessel and method of using and manufacturing the same. By using the device of the present invention, which in its fully de-deployed configuration presents a cross-sectional profile ranging from only 0.010 inches to 0.040 inches, perfusion can be restored in vascular beds heretofore considered to be relatively inaccessible for such intervention, either because of size or tortuosity. In addition, use of the device for distal protection within similar vascular beds affords protection during interventional therapies and maneuvers heretofore considered inappropriate without such protection because of the clinically devastating sequelae associated with distal showering of particulate matter during such procedures. Thus, the device and method of the present invention provide a novel system for more effectively extracting clot debris from smaller, distal coronary vessels or angioplasting and stenting the same while protecting the vascular bed distal thereto.

The device and method of the present invention is similarly effective for extracting clot and affording distal protection during interventional procedures upon the more distal, tortuous cerebral vasculature such as the M1, M2 and M3 vessels of the middle cerebral artery.

Thus, it is apparent from the foregoing that there has been provided a device and method for clot retrieval from and distal protection of blood vessels in the human body that have distinct advantages over those heretofore provided.

What is claimed is:

1. A device for distal protection within a blood vessel having a wall defining a lumen in the body comprising an elongated tubular member having proximal and distal extremities and having a longitudinal axis, an expansile member having proximal and distal extremities, the expansile member being carried by the distal extremity of the elongated tubular member and movable between radially contracted and expanded configurations, a deformable membrane at least partially covering the expansile member in the radially expanded configuration, a guide member coupled to the distal extremity of the expansile member, said guide member having proximal and distal ends and a handle assembly carried by the proximal extremity of the elongated tubular member for moving said expansile member between radially contracted and expanded configurations, said handle assembly being detachable from the proximal extremity of the elongated tubular member.

2. The device of claim 1 further comprising a guide tube carried by the distal end of the elongated tubular member and coupleable to the distal end of the guide member during movement of the expansile member between radially contracted and expanded configurations.

3. The device of claim 1 further including a lock and key mechanism for securing said handle assembly to said proximal extremity of the elongated tubular member.

4. The device of claim 1 wherein with the expansile member in the radially contracted configuration the device has a cross-sectional dimension ranging from 0.010 to 0.040 inches.

5. The device of claim 1, wherein the expansile member in the radially expanded configuration is provided with a plurality of openings through which clot material is retrieved.

6. The device of claim 5 wherein the expansile member in the radially expanded configuration is provided with an inner chamber and said deformable membrane is provided with at least a first opening overlying the proximal extremity of the expansile member whereby blood and clot material flowing in the blood vessel enters said chamber and further including a filter coupled to said expansile member through which blood exits said chamber.

7. The device of claim 6 wherein said filter includes a plurality of micropores carried by said deformable membrane.

8. The device of claim 7 wherein said plurality of micropores overlie the distal extremity of the expansile member.

9. A device for retrieval of foreign material within a blood vessel having a wall defining a lumen in the body comprising an elongated tubular member having proximal and distal extremities and having a longitudinal axis, an expansile member having proximal and distal extremities, the expansile member being carried by the distal extremity of the elongated tubular member and movable between radially contracted and expanded configurations, said expansile member in the expanded configuration assuming a substantially planar configuration, a deformable membrane at least partially covering the expansile member in the radially expanded configuration, a guide member coupled to the distal extremity of the expansile member, said guide member having proximal and distal ends and a handle assembly carried by the proximal extremity of the elongated tubular member for moving said expansile member between radially contracted and expanded configurations.

10. The device of claim 9 wherein said expansile member in the expanded configuration assumes a substantially coil-like configuration.

11. The device of claim 10 wherein said deformable membrane is provided with at least a first opening overlying the proximal extremity of the expansile member whereby blood and clot material flowing in the blood vessel enters the proximal extremity of said coil-like configuration and further including a filter coupled to said expansile member through which blood exists said coil-like configuration.

12. The device of claim 11 wherein said filter includes a plurality of micropores carried by said deformable membrane.

13. The device of claim 9 wherein said handle assembly is detachable from the proximal extremity of the elongated tubular member.

14. The device of claim 9 wherein when the expansile member assumes the radially contracted configuration the device has a cross-sectional dimension ranging from 0.010 to 0.040 inches.
